SEQUENCE LISTING (38 sequences)

SEQ ID NO: 1 - DNA, 29 bp, Artificial Sequence
  Description of Artificial Sequence Synthetic polynucleotide sequence
tttggtggtggtggttgtggtggtggtgg

SEQ ID NO: 2 - DNA, 29 bp, Artificial Sequence
  Description of Artificial Sequence Synthetic polynucleotide sequence
tttggtggtggtggttttggtggtggtgg

SEQ ID NO: 3 - DNA, 29 bp, Artificial Sequence
  Description of Artificial Sequence Synthetic polynucleotide sequence
tttggtggtggtggtggtggtggtggtgg

SEQ ID NO: 4 - DNA, 29 bp, Artificial Sequence
  Description of Artificial Sequence Synthetic polynucleotide sequence
tttggtggtggtggtttgggtggtggtgg

SEQ ID NO: 5 - DNA, 13 bp, Artificial Sequence
  Description of Artificial Sequence Synthetic polynucleotide sequence
tggtggtggtggt

SEQ ID NO: 6 - DNA, 14 bp, Artificial Sequence
  Description of Artificial Sequence Synthetic polynucleotide sequence
ggtggttgtggtgg

SEQ ID NO: 7 - DNA, 15 bp, Artificial Sequence
  Description of Artificial Sequence Synthetic polynucleotide sequence
gttgtttggggtggt

SEQ ID NO: 8 - DNA, 15 bp, Artificial Sequence
  Description of Artificial Sequence Synthetic polynucleotide sequence
ttggggggggtgggt

SEQ ID NO: 9 - DNA, 25 bp, Artificial Sequence
  Description of Artificial Sequence Synthetic polynucleotide sequence
ggttggggtgggtggggtgggtggg

SEQ ID NO: 10 - DNA, 26 bp, Artificial Sequence
  Description of Artificial Sequence Synthetic polynucleotide sequence
ggtggtggtggttgtggtggtggtgg

SEQ ID NO: 11 - DNA, 28 bp, Artificial Sequence
  Description of Artificial Sequence Synthetic polynucleotide sequence
tttggtggtggtggttgtggtggtggtg

SEQ ID NO: 12 - DNA, 28 bp, Artificial Sequence
  Description of Artificial Sequence Synthetic polynucleotide sequence
tttggtggtggtggtgtggtggtggtgg

SEQ ID NO: 13 - DNA, 29 bp, Artificial Sequence
  Description of Artificial Sequence Synthetic polynucleotide sequence
ggtggtggtggttgtggtggtggtggttt

SEQ ID NO: 14 - DNA, 32 bp, Artificial Sequence
  Description of Artificial Sequence Synthetic polynucleotide sequence
ggtggttgtggtggttgtggtggttgtggtgg

SEQ ID NO: 15 - DNA, 32 bp, Artificial Sequence
  Description of Artificial Sequence Synthetic polynucleotide sequence
tttggtggtggtggttgtggtggtggtggttt

SEQ ID NO: 16 - DNA, 56 bp, Artificial Sequence
  Description of Artificial Sequence Synthetic polynucleotide sequence
ggtggtggtggttgtggtggtggtggttgtggtggtggtggttgtggtggtggtgg

SEQ ID NO: 17 - DNA, 26 bp, Artificial Sequence
  Description of Artificial Sequence Synthetic polynucleotide sequence
cctcctcctccttctcctcctcctcc

SEQ ID NO: 18 - DNA, 29 bp, Artificial Sequence
  Description of Artificial Sequence Synthetic polynucleotide sequence
tttcctcctcctccttctcctcctcctcc

SEQ ID NO: 19 - DNA, 24 bp, Artificial Sequence
  Description of Artificial Sequence Synthetic polynucleotide sequence
ttagggttagggttagggttaggg

SEQ ID NO: 20 - DNA, 11 bp, Artificial Sequence
  Description of Artificial Sequence Synthetic polynucleotide sequence
ggtggtggtgg

SEQ ID NO: 21 - DNA, 14 bp, Artificial Sequence
  Description of Artificial Sequence Synthetic polynucleotide sequence
ggtggttgtggtgg

SEQ ID NO: 22 - DNA, 15 bp, Artificial Sequence
  Description of Artificial Sequence Synthetic polynucleotide sequence
ggttggtgtggttgg

SEQ ID NO: 23 - DNA, 10 bp, Artificial Sequence
  Description of Artificial Sequence Synthetic polynucleotide sequence
gggttttggg

SEQ ID NO: 24 - DNA, 20 bp, Artificial Sequence
  Description of Artificial Sequence Synthetic polynucleotide sequence
ggttttggttttggttttgg

SEQ ID NO: 25 - DNA, 15 bp, Artificial Sequence
  Description of Artificial Sequence Synthetic polynucleotide sequence
ggttggtgtggttgg

SEQ ID NO: 26 - DNA, 12 bp, Artificial Sequence
  Description of Artificial Sequence Synthetic polynucleotide sequence
ggggttttgggg

SEQ ID NO: 27 - DNA, 10 bp, Artificial Sequence
  Description of Artificial Sequence Synthetic polynucleotide sequence
gggttttggg

SEQ ID NO: 28 - DNA, 28 bp, Artificial Sequence
  Description of Artificial Sequence Synthetic polynucleotide sequence
ggggttttggggttttggggttttgggg

SEQ ID NO: 29 - DNA, 24 bp, Artificial Sequence
  Description of Artificial Sequence Synthetic polynucleotide sequence
ttggggttggggttggggttgggg

SEQ ID NO: 30 - DNA, 16 bp, Artificial Sequence
  Description of Artificial Sequence Synthetic polynucleotide sequence
gggtgggtgggtgggt

SEQ ID NO: 31 - DNA, 26 bp, Artificial Sequence
  Description of Artificial Sequence Synthetic polynucleotide sequence
ggttttggttttggttttggttttgg

SEQ ID NO: 32 - DNA, 29 bp, Artificial Sequence
  Description of Artificial Sequence Synthetic polynucleotide sequence
tttcctcctcctccttctcctcctcctcc

SEQ ID NO: 33 - DNA, 26 bp, Artificial Sequence
  Description of Artificial Sequence Synthetic polynucleotide sequence
cctcctcctccttctcctcctcctcc

SEQ ID NO: 34 - DNA, 6 bp, Artificial Sequence
  Description of Artificial Sequence Synthetic polynucleotide sequence
tggggt

SEQ ID NO: 35 - DNA, 7 bp, Artificial Sequence
  Description of Artificial Sequence Synthetic polynucleotide sequence
gcatgct

SEQ ID NO: 36 - DNA, 11 bp, Artificial Sequence
  Description of Artificial Sequence Synthetic polynucleotide sequence
gcggtttgcgg

SEQ ID NO: 37 - DNA, 4 bp, Artificial Sequence
  Description of Artificial Sequence Synthetic polynucleotide sequence
tagg

SEQ ID NO: 38 - DNA, 23 bp, Artificial Sequence
  Description of Artificial Sequence Synthetic polynucleotide sequence
ggggttggggtgtggggttgggg

(19) United States
(12) Patent Application Publication
(10) Pub. No.: US 2023/0181769 A1
(43) Pub. Date: Jun. 15, 2023

(54) ANTI-NUCLEOLIN AGENT-PEG-CONJUGATED NANOPARTICLES

(71) Applicant: University of Louisville Research Foundation, Inc., Louisville, KY (US)

(72) Inventors: Nicholas C. Allen, Louisville, KY (US); Paula J. Bates, Louisville, KY (US); Martin G. O'Toole, Louisville, KY (US)

(21) Appl. No.: 17/641,787

(22) PCT Filed: Sep. 10, 2020
(86) PCT No.: PCT/US2020/050261

§ 371 Date: Mar. 9, 2022

Related U.S. Application Data
(60) Provisional application No. 62/898,527, filed on Sep. 10, 2019.

(51) Int. Cl. A61K 49/18; A61K 47/69; A61K 47/60; A61P 35/00

(52) U.S. Cl. CPC A61K 49/186; A61K 47/6929; A61K 47/60; A61P 35/00

(57) ABSTRACT

A composition comprises an anti-nucleolin agent and PEG conjugated to nanoparticles. Preferably the nanoparticles have an average diameter of 1 to 50 nm. Preferably, the nanopanicles comprise at least one inorganic material selected from the group consisting of metals, elements and oxides.

### BACKGROUND

Nucleolin [37] is an abundant, non-ribosomal protein of the nucleolus, the site of ribosomal gene transcription and packaging of pre-ribosomal RNA. This 710 amino acid phosphoprotein has a multi-domain structure consisting of a histone-like N-terminus, a central domain containing four RNA recognition motifs and a glycine/arginine-rich C-terminus, and has an apparent molecular weight of 110 kD. While nucleolin is found in every nucleated cell, the expression of nucleolin on the cell surface has been correlated with the presence and aggressiveness of neoplastic cells [38].

The correlation of the presence of cell surface nucleolin with neoplastic cells has been used for methods of determining the neoplastic state of cells by detecting the presence of nucleolin on the plasma membranes [38]. This observation has also provided new cancer treatment strategies based on administering compounds that specifically target nucleolin [39].

Nucleic acid aptamers are short synthetic oligonucleotides that fold into unique three-dimensional structures that can be recognized by specific target proteins. Thus, their targeting mechanism is similar to monoclonal antibodies, but they may have substantial advantages over these, including more rapid clearance in vivo, better tumor penetration, non-immunogenicity, and easier synthesis and storage.

Guanosine-rich oligonucleotides (GROs) designed for triple helix formation are known for binding to nucleolin. This ability to bind nucleolin has been suggested to cause their unexpected ability to effect antiproliferation of cultured prostate carcinoma cells [40]. The antiproliferative effects are not consistent with a triplex-mediated or an antisense mechanism, and it is apparent that GROs inhibit proliferation by an alternative mode of action. It has been surmised that GROs, which display the propensity to form higher order structures containing G-quartets, work by an aptamer mechanism that entails binding to nucleolin due to a shape-specific recognition of the GRO structure; the binding to cell surface nucleolin then induces apoptosis. The antiproliferative effects of GROs have been demonstrated in cell lines derived from prostate (DU145), breast (MDA-MB-231, MCF-7), or cervical (HeLa) carcinomas and correlates with the ability of GROs to bind cell surface nucleolin [40].

AS1411 (GRO26B, SEQ ID NO:10), a GRO nucleolin-binding DNA aptamer that has antiproliferative activity against cancer cells with little effect on non-malignant cells, was previously developed. AS1411 uptake appears to occur by macropinocytosis in cancer cells, but by a nonmacropinocytic pathway in nonmalignant cells, resulting in the selective killing of cancer cells, without affecting the viability of nonmalignant cells [41]. AS1411 was the first anticancer aptamer tested in humans and results from clinical trials of AS1411 (including Phase II studies in patients with renal cell carcinoma or acute myeloid leukemia) indicate promising clinical activity with no evidence of serious side effects. Despite a few dramatic and durable clinical responses, the overall rate of response to AS1411 was low, possibly due to the low potency of AS1411.

Anti-nucleolin agents conjugated to particles, such as aptamers conjugated to gold nanoparticles, have an antiproliferative effect on cancer and tumors. See International Application, International Publication Number WO 2012/167173, entitled “ANTI-NUCLEOLIN AGENT-CONJUGATED NANOPARTICLES”, filed 1 Jun. 2012, to Bates et al. Aptamer conjugated gold nanoparticles, in particular, have a similar or greater antiproliferative effect than the aptamer (anti-nucleolin oligonucleotide) alone, demonstrating similar effects at only 1/10 to 1/100 the dosage. Furthermore, these same agents, preferably having a fluorescent dye conjugated to the particle or attached to the anti-nucleolin agent, may also be used as imaging agents, both in vivo and ex vivo. See also [49].

Anti-nucleolin agents conjugated to particles comprising metals, such as aptamers conjugated to gold nanoparticles, are effective radio-sensitizers for treating cancer. The nanoparticles are selectively taken-up by cancer cells and enhance the effects of RT on those cells. This enhances the effectiveness of RT, and/or allows a low effective dose of radiation to be used during RT. Furthermore, the nanoparticles may optionally also contain gadolinium, enhancing the relaxivity (speed up the relaxation rate) of nearby water molecules during an MRI scan and contribute to an increase in contrast when present in the sample being scanned. In addition, both the gold nanoparticles and the Gd ions enhance the absorption and scattering of X-rays, and increase the contrast when present in the sample being scanned or imaged using X-ray based imaging techniques, such as CT scanning. The combination of anti-nucleolin agents (cancer targeting) and gadolinium (MRI contrast enhancement) combine to result in a cancer-targeting MRI-contrast agent. Furthermore, the combination of anti-nucleolin agents (cancer targeting) and gold nanoparticle (X-ray contrast enhancement) and optionally gadolinium (MRI contrast enhancement and X-ray contrast enhancement) combine to result in a cancer-targeting MRI-contrast and X-ray (CT scan) contrast agent. See International Application, International Publication Number WO 2016/179394, entitled “ANTI-NUCLEOLIN AGENT-CONJUGATED NANOPARTICLES AS RADIO-SENSITIZERS AND MRI AND/OR X-RAY CONTRAST AGENTS”, filed 5 May 2016, to Bates et al.

AS1411 has also been used as a cancer targeting agent for selective treatment of cancer cells with an anti-cancer drug or complex. For example, drug containing PLGA nanoparticles have been functionalized with AS1411 and conjugated to poly(ethylene glycol) (PEG) to specifically target cancer cells, where the PLGA then releases the drug as it is degraded within the cells [46, 48].

Poly(ethylene glycol) (PEG) modification of drugs, often referred to as PEGylation, has been used extensively to improve solubility and in vivo stability by preventing the rapid renal clearance of the drug. In addition, PEGylation helps hide the drug from the immune system, so that the patient will have less likelihood of developing an immune response neutralizing the effectiveness of the drug over the course of treatment (which is particularly problematic with high-molecular weight drugs such as bioengineered enzymes and antibodies). Furthermore, PEGylation results in enhanced permeability and retention (EPR) effects in tumors, taking advantage of the leaky vasculature of tumors and lack of effective lymphatic drainage to enhance effective tumor targeting. For example, PEG modified with a chelating group, chelated to Cu ions and then complexed with AS1411, has been studied for targeting tumor cells [47].

Glioblastomas (GBM) are the most frequent adult primary brain tumor and typically have a prognosis of 14 months with 5-year survival rate <5% [20-24]. The most promising treatment is surgery combined with chemo and radio therapies [25, 26]. Full resection is not always available, and treatment with chemo/radio therapies have potential toxicity [27-30]. GBM is characterized by aberrant modifications of signaling pathways, allowing for the hijacking of proliferation, migration, and survival of the GBM [24, 31-34].

### SUMMARY

In a first aspect, the present invention is a composition, comprising an anti-nucleolin agent and PEG conjugated to nanoparticles. The nanoparticles have an average diameter of 1 to 50 nm.

In a second aspect, the present invention is a composition, comprising an anti-nucleolin agent and PEG conjugated to nanoparticles. The nanoparticles comprise at least one inorganic material selected from the group consisting of metals, elements and oxides.

In a third aspect, the present invention is a method of treating cancer, comprising administering an effective amount of any of the preceding compositions, to a patient in need thereof.

In a fourth aspect, the present invention is a method of treating cancer, comprising administering an effective amount of any of the preceding compositions, to a patient in need thereof, followed by radiation therapy.

In a fifth aspect, the present invention is a method of imaging cancer by MRI and/or X-ray imaging techniques in vivo, comprising administering any of the preceding compositions, to a subject and forming an image of the composition present in the subject by MRI and/or an X-ray imaging technique.

### Definitions

The term “conjugated” means “chemically bonded to”.

The term “anti-nucleolin oligonucleotides” refers to an oligonucleotide that binds to nucleolin.

The term “equivalent aptamer concentration” refers to the concentration of anti-nucleolin oligonucleotide present in the conjugate.

Poly(ethylene glycol), also referred to as PEG, may be conjugated to a drug, nanoparticles or other substances. When conjugated to PEG, they are often referred to as PEGylated, and will contain the —(CH₂CH₂O)— moiety. The molecular weight of PEG is understood to be the number-average molecular weight (Mn) of PEG prior to conjugation, preferably as labelled by the manufacturer or seller, unless otherwise specified.

Tumors and cancers include solid, dysproliferative tissue changes and diffuse tumors. Examples of tumors and cancers include melanoma, lymphoma, plasmocytoma, sarcoma, glioma, glioblastoma, thymoma, leukemia, breast cancer, prostate cancer, colon cancer, liver cancer, esophageal cancer, brain cancer, lung cancer, ovary cancer, endometrial cancer, bladder cancer, kidney cancer, cervical cancer, hepatoma, and other neoplasms. For more examples of tumors and cancers, see, for example Stedman [42].

“Treating a tumor” or “treating a cancer” means to significantly inhibit growth and/or metastasis of the tumor or cancer, and/or killing cancer cells. Growth inhibition can be indicated by reduced tumor volume or reduced occurrences of metastasis. Tumor growth can be determined, for example, by examining the tumor volume via routine procedures (such as obtaining two-dimensional measurements with a dial caliper). Metastasis can be determined by inspecting for tumor cells in secondary sites or examining the metastatic potential of biopsied tumor cells in vitro.

A “chemotherapeutic agent” is a chemical compound that can be used effectively to treat cancer in humans.

A “pharmaceutically acceptable carrier” includes any and all solvents, dispersion media, coatings, antibacterial and antifungal agents, isotonic and absorption delaying agents which are compatible with pharmaceutical administration. Preferred examples of such carriers or diluents include water, saline, Ringer's solutions and dextrose solution. Supplementary active compounds can also be incorporated into the compositions.

“Medicament,” “therapeutic composition” and “pharmaceutical composition” are used interchangeably to indicate a compound, matter, mixture or preparation that exerts a therapeutic effect in a subject.

“Antibody” is used in the broadest sense and refers to monoclonal antibodies, polyclonal antibodies, multispecific antibodies, antibody fragments and chemically modified antibodies, where the chemical modification does not substantially interfere with the selectivity and specificity of the antibody or antibody fragment.

An “anti-nucleolin agent” includes any molecule or compound that interacts with nucleolin. Such agents include for example anti-nucleolin antibodies, aptamers such GROs and nucleolin targeting proteins.

“X-ray based imaging techniques” include all imaging techniques which use X-rays to form an image, directly or indirectly, including for example CT scans (also called X-ray computed tomography or computerized axial tomography scan (CAT scan)).

### BRIEF DESCRIPTION OF THE DRAWINGS

FIG. 1 is a cartoon representation of a co-conjugated gold nanoparticle.

FIG. 2A is a graph of the zeta potential (mV) of various ratios of AS1411 to PEG. FIG. 2A represents an average of two independent particle syntheses and the values are reported as mean±standard deviation.

FIG. 2B is a graph of the hydrodynamic size (nm) of various ratios of AS1411 to PEG. FIG. 2B represents an average of two independent particle syntheses and the values are reported as mean±standard deviation.

FIG. 2C is a graph of the polydispersity indices of various ratios of AS1411 to PEG. FIG. 2C represents an average of two independent particle syntheses and the values are reported as mean±standard deviation.

FIG. 2D is a graph of the estimated number of AS1411 molecules loaded onto gold nanoparticles based on DTT separation of conjugated oligonucleotides and UV-VIS spectral analysis before and after separation. FIG. 2D represents an average of two independent particle syntheses and the values are reported as mean±standard deviation.

FIG. 2E is a graph of the UV-VIS analysis of particle syntheses showing particle absorptions from 210-450 nm.

FIG. 2F is a graph of the UV-VIS analysis of particle syntheses showing particle absorptions from 450-1000 nm with the surface plasmon resonance wavelengths of gold.

FIG. 3 is a graph showing the in-vitro metabolic activity of U87MGs after treatment with co-conjugated AuNP ratio particles. Data presented as mean±std. dev (n=3). Bars represent absolute XTT absorbance of treated U87MGs relating proportionally to U87MG viability. Statistically significant cytotoxicity of 6× and 9×particles determined by two-way ANOVA analysis, α=0.05, * indicates p<0.0001 when compared to 0 and 10 μM AS groups. Significance determined using Graph Pad Prism software.

FIG. 4 is a graph showing the EC50 analysis of significant 6× and 9×particles. Curves represent non-linear regression line of best fit for each group. EC50 values determined to be 1303 nM (r2=0.8722) and 212 nM (r2=0.9181) oligo for 9× and 6×particles, respectively. Analysis completed with Graph Pad Prism software.

FIG. 5 illustrates 10×brightfield images of ratio particle treated U87MGs showing cellular morphology and density after 72 hours. Images were acquired using Nikon epifluorescent microscope and NIS elements software. AuNP treatments represent ˜1000 nM oligo concentrations except for 10 μM AS1411 condition. Scale bar indicates 100 microns.

FIG. 6 is a graph showing the in-vitro metabolic activity of U87MGs after treatment with 9×AS/CRO AuNPs. Data presented as mean±std. dev (n=5). Bars represent absolute XTT absorbance of treated U87MGs relating proportionally to U87MG viability. Statistically significant specificity of 9×particles determined by two-way ANOVA analysis, α=0.05, * indicates p<0.001 when 9×compared to equivalent CRO group. Significance determined using Graph Pad Prism software.

FIG. 7 is a graph showing EC50 analysis of 9×co-conjugated AuNP particles. Curve represent non-linear regression line of best fit. EC50 value was determined to be 935 nM oligo (r2=0.7015) CRO curve not obtainable. Analysis completed with Graph Pad Prism software.

### DETAILED DESCRIPTION

The present invention makes use of anti-nucleolin agents and PEG, conjugated to particles, such as an aptamer and PEG conjugated to gold nanoparticles, having an antiproliferative effect on cancer and tumors. The PEG/anti-nucleolin agent conjugates may also be referred to as co-conjugates. The addition of PEG as a co-conjugate may enhance the pharmacokinetics of the anti-nucleolin agents conjugated to nanoparticles, further improving efficacy in vivo, such as by increasing circulatory half-life, or avoid accumulation is organs, such as the liver and/or the lungs. Furthermore, aptamer/PEG conjugated gold nanoparticles in particular have a similar or greater antiproliferative effect than the aptamer (anti-nucleolin oligonucleotide) alone, or even aptamer conjugated gold nanoparticles, and may demonstrate similar effects at only 1/10 to 1/100 the dosage. In addition, these same agents, preferably having a fluorescent dye conjugated to the particle or attached to the anti-nucleolin agent or PEG, may also be used as imaging agents, both in vivo and ex vivo. They may also be used as agents to enhance radiation therapy (RT) and as contrast agent for X-rays and CT scans. Lastly, if also conjugated to gadolinium, they may be used to enhance contrast in MRI scans.

The molar ratio between the PEG and anti-nucleolin agent conjugated to the nanoparticles may be varied. For example, the ratio of anti-nucleolin agent PEG may be 1:1, 2:1, 3:1, 4:1, 5:1, 6:1, 7:1, 8:1, 9:1, 10:1, 11:1 or 12:1. Alternatively, the ratio of PEG:anti-nucleolin agent may be 1:1, 2:1, 3:1, 4:1, 5:1, 6:1, 7:1, 8:1, 9:1, 10:1, 11:1 or 12:1. Preferably, the anti-nucleolin agent is AS1411. Preferably, the nanoparticles are gold nanoparticles having a diameter of 4, 5, 10, 15, 20, or 100 nm. For treating glioblastoma or other brain cancer, preferably the nanoparticles have a diameter of 4, 5 or 10 nm, to enhance penetration through the blood-brain barrier.

When conjugating an anti-nucleolin agent (such as AS1411) and/or PEG to a nanoparticle (such as gold nanoparticles), typically the surface of the nanoparticle is saturated with the agents conjugated to the surface. Therefore, when the mixture of agents (such as AS1411 and PEG) is formed, the ratio of the agents is controlled, and this controls the ratio of the agents conjugated to the nanoparticles, and the number of each agent on each nanoparticle. For example, in the case of 4 nm gold nanoparticles conjugated with AS1411 and PEG, the total number of agent molecules which are conjugated will be 12, so the sum of AS1411 and PEG molecules conjugated to the nanoparticle will be 12. The nomenclature used to identify the ratio of AS1411:PEG is based on the total number of AS1411 molecules conjugated to the nanoparticle. In the case of a 4 nm gold nanoparticle, “12×” means 12 AS1411 molecules, and an AS1411:PEG ratio of 12:0; similarly, “9×”, “6×”, “3×” and “0×”, have 9, 6, 3 and 0 AS1411 molecules, respectively, and a AS1411:PEG ratio of 9:3, 6:6, 3:9 and 0:12, respectively (or alternatively 3:1, 1:1, 1:3 and 0, respectively). With a larger gold nanoparticle, such as a 5 nm or 10 nm gold nanoparticle, the total amount agents which may be conjugated is greater, so “9×” would imply a smaller AS1411:PEG ratio. Although a distribution of conjugates form during synthesis, those nanoparticles which deviate from the most prevalent conjugation ratio may be separated by “salting out,” taking advantage of the different zeta potentials of nanoparticles with different conjugation ratios.

Any molecular weight of PEG may be used. Preferably, the PEG molecular weight is 200 to 20,000, more preferably 1000 to 10,000, including 2000, 3000, 4000, 5000, 6000, 7000, 8000 and 9000, and values in between. It is understood that these molecular weight values are number-average molecular weights that represent the value as expressed to the significant figures of the accuracy of the measurement, and that such compositions have a distribution of PEG molecules about the reported molecular weight.

Radiation treatment (RT) may be by any form of radiation, such as X-rays (for example megavolt energy X-rays), Brachy therapy, proton radiation, and neutron radiation. Also possible is to use doses and/or energy of RT that would normally be considered subclinical; because the anti-nucleolin agent-PEG-conjugated nanoparticles enhance the effectiveness of RT, the dosages are effective to kill or reduce the growth of cancer cells and tumors.

Anti-nucleolin agent-PEG-conjugated nanoparticles which contain gadolinium are effective MRI contrast agents, and may also be used to image cancer cells, including individual cancer cells. For example, the anti-nucleolin agent-PEG-conjugated nanoparticles which contain gadolinium may be administered to a patient to determine if cancer cells are present in lymph nodes, thus avoiding the removal of lymph node for the sole purpose of determining if they contain cancer cells. Another use can be to avoid the need for a biopsy. The anti-nucleolin agent-PEG-conjugated nanoparticles which contain gadolinium may be administered to a patient to determine if cancer is present in a lump, has metastasized to other location in the body, or to determine if all cancer from a tumor has been removed during surgery.

Anti-nucleolin agent-PEG-conjugated nanoparticles which optionally contain gadolinium are effective X-ray contrast agents, and may also be used to image cancer cells, including individual cancer cells. For example, the anti-nucleolin agent-PEG-conjugated nanoparticles which optionally contain gadolinium may be administered to a patient to determine if cancer cells are present in lymph nodes, thus avoiding the removal of lymph node for the sole purpose of determining if they contain cancer cells. Another use can be to avoid the need for a biopsy. The anti-nucleolin agent-PEG-conjugated nanoparticles which optionally contain gadolinium may be administered to a patient to determine if cancer is present in a lump, has metastasized to other location in the body, or to determine if all cancer from a tumor has been removed during surgery.

Anti-nucleolin agents include (i) aptamers, such as GROs; (ii) anti-nucleolin antibodies; and (iii) nucleolin targeting proteins. Examples of aptamers include guanosine-rich oligonucleotides (GROs). Examples of suitable oligonucleotides and assays are also given in Miller et al. [43]. Characteristics of GROs include:

(1) having at least 1 GGT motif,

(2) preferably having 4-100 nucleotides, although GROs having many more nucleotides are possible,

(3) optionally having chemical modifications to improve stability.

Especially useful GROs form G-quartet structures, as indicated by a reversible thermal denaturation/renaturation profile at 295 nm [40]. Preferred GROs also compete with a telomere oligonucleotide for binding to a target cellular protein in an electrophoretic mobility shift assay [40] In some cases, incorporating the GRO nucleotides into larger nucleic acid sequences may be advantageous; for example, to facilitate binding of a GRO nucleic acid to a substrate without denaturing the nucleolin-binding site. Examples of oligonucleotides are shown in Table 1; preferred oligonucleotides include SEQ ID NOs: 1-7; 9-16; 19-30 and 31 from Table 1.

TABLE 1
Non-antisense GROs that bind nucleolin
and non-binding controls^{1,2,3}.

| | | SEQ |
|---|---|---|
| GRO | Sequence | ID NO: |
| GRO29A¹ | tttggtggtg gtggttgtgg tggtggtgg | 1 |
| GRO29-2 | tttggtggtg gtggttttgg tggtggtgg | 2 |
| GRO29-3 | tttggtggtg gtggtggtgg tggtggtgg | 3 |
| GRO29-5 | tttggtggtg gtggtttggg tggtggtgg | 4 |
| GRO29-13 | tggtggtggt ggt | 5 |
| GRO14C | ggtggttgtg gtgg | 6 |
| GRO15A | gttgtttggg gtggt | 7 |
| GRO15B² | ttgggggggg tgggt | 8 |
| GRO25A | ggttggggtg ggtggggtgg gtggg | 9 |
| GRO26B¹ | ggtggtggtg gttgtggtgg tggtgg | 10 |
| GRO28A | tttggtggtg gtggttgtgg tggtggtg | 11 |
| GRO28B | tttggtggtg gtggtgtggt ggtggtgg | 12 |
| GRO29-6 | ggtggtggtg gttgtggtgg tggtggttt | 13 |
| GRO32A | ggtggttgtg gtggttgtgg tggttgtggt gg | 14 |
| GRO32B | tttggtggtg gtggttgtgg tggtggtggt tt | 15 |
| GRO56A | ggtggtggtg gttgtggtgg tggtggttgt | 16 |
| | ggtggtggtg gttgtggtgg tggtgg | |
| CRO | cctcctcctc cttctcctcc tcctcc | 17 |
| CRO-2 | tttcctcctc ctccttctcc tcctcctcc | 18 |
| GRO A | ttagggttag ggttagggtt aggg | 19 |
| GRO B | ggtggtggtg g | 20 |
| GRO C | ggtggttgtg gtgg | 21 |
| GRO D | ggttggtgtg gttgg | 22 |
| GRO E | gggttttggg | 23 |
| GRO F | ggttttggtt ttggttttgg | 24 |
| GRO G¹ | ggttggtgtg gttgg | 25 |
| GRO H¹ | ggggttttgg gg | 26 |
| GRO I¹ | gggttttggg | 27 |
| GRO J¹ | ggggttttgg ggttttgggg ttttgggg | 28 |
| GRO K¹ | ttggggttgg ggttggggtt gggg | 29 |
| GRO L¹ | gggtgggtgg gtgggt | 30 |
| GRO M¹ | ggttttggtt ttggttttgg ttttgg | 31 |
| GRO N² | tttcctcctc ctccttctcc tcctcctcc | 32 |
| GRO O² | cctcctcctc cttctcctcc tcctcc | 33 |
| GRO P² | tggggt | 34 |
| GRO Q² | gcatgct | 35 |
| GRO R² | geggtttgcg g | 36 |
| GRO S² | tagg | 37 |
| GRO T² | ggggttgggg tgtggggttg ggg | 38 |

¹Indicates a good plasma membrane nucleolin-binding GRO.
²lndicates a nucleolin control (non-plasma membrane
nucleolin binding).
³GRO sequence without ¹ or ² designations have some
anti-proliferative activity.

Any antibody that binds nucleolin may also be used. In certain instances, monoclonal antibodies are preferred as they bind single, specific and defined epitopes. In other instances, however, polyclonal antibodies capable of interacting with more than one epitope on nucleolin may be used. Many anti-nucleolin antibodies are commercially available, and are otherwise easily made. See, for example, US Patent Application Publication No. US 2013/0115674 to Sutkowski et al. Table 2 lists a few commercially available anti-nucleolin antibodies.

TABLE 2
commercially available anti-nucleolin antibodies

| | | Antigen |
|---|---|---|
| Antibody | Source | source |
| p7-1A4 Mouse monoclonal | Developmental Studies | Xenopus laevis |
| antibody (mAb) | Hybridoma Bank | oocytes |
| Sc-8031 mouse mAb | Santa Cruz Biotech | human |
| Sc-9893 goat polyclonal | Santa Cruz Biotech | human |
| Ab (pAb) | | |
| Sc-9892 goat pAb | Santa Cruz Biotech | human |
| Clone 4E2 mouse mAb | MBL International | human |
| Clone 3G4B2 mouse mAb | Upstate | dog |
| | Biotechnology | (MDCK cells) |
| Nucleolin, Human | MyBioSource | human |
| (mouse mAb) | | |
| Purified anti-Nucleolin- | BioLegend | human |
| Phospho, Thr76/Thr84 | | |
| (mouse mAb) | | |
| Rabbit Polyclonal | Novus Biologicals | human |
| Nucleolin Antibody | | |
| Nucleolin (NCL, C23, | US Biological | human |
| FLJ45706, FLJ59041, | | |
| Protein C23) Mab Mo xHu | | |
| Nucleolin (NCL, Nucl, | US Biological | human |
| C23, FLJ45706, Protein | | |
| C23) Pab Rb xHu | | |
| Mouse Anti-Human | Cell Sciences | human |
| Nucleolin Phospho- | | |
| Thr76/Thr84 Clone | | |
| 10C7 mAb | | |
| Anti-NCL/Nucleolin | LifeSpan | human |
| (pAb) | Biosciences | |
| NCL purified MaxPab | Abnova | human |
| mouse polyclonal | | |
| antibody (B02P) | | |
| NCL purified MaxPab | Abnova | human |
| rabbit polyclonal | | |
| antibody (D01P) | | |
| NCL monoclonal | Abnova | human |
| antibody, clone | | |
| 10C7 (mouse mAb) | | |
| Nucleolin Monoclonal | Enzo Life Sciences | human |
| Antibody (4E2) | | |
| (mouse mAb) | | |
| Nucleolin, Mouse | Life Technologies | human |
| Monoclonal Antibody | Corporation | |
| NCL Antibody (Center | Abgent | human |
| E443) (rabbit pAb) | | |
| Anti-Nucleolin, clone | EMD Millipore | human |
| 3G4B2 (mouse mAb) | | |
| NCL (rabbit pAb) | Proteintech Group | human |
| Mouse Anti-Nucleolin | Active Motif | human |
| Monoclonal Antibody, | | |
| Unconjugated, Clone | | |
| 3G4B20 | | |
| Nsr1p - mouse monoclonal | EnCor Biotechnology | human |
| Nucleolin (mouse mAb) | Thermo Scientific | human |
| | Pierce Products | |
| Nucleolin [4E2] antibody | GeneTex | human |
| (mouse mAb) | | |

Nucleolin targeting proteins are proteins, other than antibodies, that specifically and selectively bind nucleolin. Examples include ribosomal protein S3, tumor-homing F3 peptides [44,45] and myosin H9 (a non-muscle myosin that binds cell surface nucleolin of endothelial cells in angiogenic vessels during tumorigenesis).

Anti-nucleolin agents may be conjugated to particles made of a variety of materials solid materials, including (1) metals and elements; (2) oxides; (3) semiconductors; and (4) polymers. Metals and elements, preferably non-magnetic metals and elements, include gold, silver, palladium, iridium, platinum and alloys thereof; elements include silicon, boron and carbon (such as diamond, graphene and carbon nanotubes), and solid compounds thereof. Oxides include titanium dioxide, silicon dioxide, zinc oxide, iron oxide, zirconium oxide, magnesium oxide, aluminum oxide and complex oxides thereof, such as barium titanate. Semiconductors include quantum dots, zinc sulfide, silicon/germanium alloys, boron nitride, aluminum nitride, and solid solutions thereof. Polymers include polyethylenes, polystyrenes, polyacrylamide, polyacrylates and polymethacrylates, and polysiloxanes. Preferably, the particles are non-toxic. The particles are preferably nanoparticles having an average particle diameter of 1-100 nm, more preferably 1-50 nm, including 4, 5, 6, 7, 8, 9, 10, 12, 15, 18, 20, 25, 30, 35, 40, 45, 50, 55, 60, 65, 70, 75, 80, 85, 90 and 95 nm.

Oligonucleotides and proteins have been attached to solid materials, such as metals and elements, oxides, semiconductors and polymers, by a variety of techniques. These same techniques may be used to attached anti-nucleolin agents to particles. Further attachment of dyes to the anti-nucleolin agent-PEG conjugated nanoparticles (conjugates), such as cyanine dyes, allows the conjugates to be used as imaging agents, both in vivo and ex vivo.

Anti-nucleolin agent-PEG conjugated nanoparticles may be used to formulate a pharmaceutical composition for treating cancer and tumors, and targeting cancer cells expressing cell surface nucleolin, by forming mixtures off the anti-nucleolin agent-PEG conjugated nanoparticles and a pharmaceutically acceptable carrier, such as a pharmaceutical composition. Methods of treating cancer in a subject include administering a therapeutically effective amount of an anti-nucleolin agent-PEG conjugated nanoparticles.

Particularly preferred compositions are aptamers conjugated to gold nanoparticles. Gold nanoparticles (GNPs) exhibit low toxicity, versatile surface chemistry, light absorbing/scattering properties, and tunable size. Aptamers effectively cap gold particles and prevent aggregation, are safe, stable, easy to synthesize, and non-immunogenic. Aptamer conjugated GNPs offer many advantages over alternative approaches, such as enhanced antiproliferative activity in cancer cells over AS1411 alone and improved efficacy in vivo, causing durable regression of established breast cancer xenografts in mice, without evidence of side effects. Aptamer conjugated GNP are highly selective for cancer cells over normal cells, and when attached to cyanine dyes are excellent imaging agents, for example Cy2, Cy3, Cy5, Cy®5.5, Cy7, Alexa Fluor® 680, Alexa Fluor® 750, IRDye® 680, and IRDyee® 800CW (LI-COR Biosciences, Lincoln, Nebr.). Aptamer conjugated GNP may be used as an imaging agent, and may be administered as compositions which further contain a pharmaceutically acceptable carrier. The imaging agent may be administered to a subject in a method of imaging cancer in vivo, to form an image of the imaging agent present in the subject.

The amounts and ratios of compositions described herein are all by weight, unless otherwise stated. Accordingly, the number of anti-nucleolin agents per nanoparticle may vary when the weight of the nanoparticle varies, even when the equivalent anti-nucleolin agent concentration (or equivalent aptamer concentration) is otherwise the same. For example, the number of anti-nucleolin agent molecules per nanoparticle may vary from 2 to 10,000, or 10 to 1000, including 20, 30, 40, 50, 60, 70, 80, 90, 100, 200, 300, 400, 500, 600, 700, 800 and 900.

A pharmaceutical composition is formulated to be compatible with its intended route of administration, including intravenous, intradermal, subcutaneous, oral, inhalation, transdermal, transmucosal, and rectal administration. Solutions and suspensions used for parenteral, intradermal or subcutaneous application can include a sterile diluent, such as water for injection, saline solution, polyethylene glycols, glycerin, propylene glycol or other synthetic solvents; antibacterial agents such as benzyl alcohol or methyl parabens; antioxidants such as ascorbic acid or sodium bisulfite; buffers such as acetates, citrates or phosphates, and agents for the adjustment of tonicity such as sodium chloride or dextrose. The pH can be adjusted with acids or bases, such as hydrochloric acid or sodium hydroxide.

Pharmaceutical compositions suitable for injection include sterile aqueous solutions or dispersions for the extemporaneous preparation of sterile injectable solutions or dispersion. For intravenous administration, suitable carriers include physiological saline, bacteriostatic water, CREMOPHOR EL® (BASF; Parsippany, N.J.) or phosphate buffered saline (PBS). In all cases, the composition must be sterile and should be fluid so as to be administered using a syringe. Such compositions should be stable during manufacture and storage and are preferably preserved against contamination from microorganisms such as bacteria and fungi. The carrier can be a dispersion medium containing, for example, water, polyol (such as glycerol, propylene glycol, and liquid polyethylene glycol), and other compatible, suitable mixtures. Various antibacterial and anti-fungal agents, for example, parabens, chlorobutanol, phenol, ascorbic acid, and thimerosal, can contain microorganism contamination. Isotonic agents such as sugars, polyalcohols, such as mannitol, sorbitol, and sodium chloride can be included in the composition. Compositions that can delay absorption include agents such as aluminum monostearate and gelatin.

Sterile injectable solutions can be prepared by incorporating the active agents, and other therapeutic components, in the required amount in an appropriate solvent with one or a combination of ingredients as required, followed by sterilization. Methods of preparation of sterile solids for the preparation of sterile injectable solutions include vacuum drying and freeze-drying to yield a solid.

The pharmaceutical composition described herein may further comprise other therapeutically active compounds, and/or may be used in conjunction with physical techniques as noted herein which are suitable for the treatment of cancers and tumors. Examples of commonly used therapeutically active compounds include vinorelbine (Navelbine®), mytomycin, camptothecin, cyclyphosphamide (Cytoxin®), methotrexate, tamoxifen citrate, 5-fluorouracil, irinotecan, doxorubicin, flutamide, paclitaxel (Taxol®), docetaxel, vinblastine, imatinib mesylate (Gleevec®), anthracycline, letrozole, arsenic trioxide (Trisenox®), anastrozole, triptorelin pamoate, ozogamicin, irinotecan hydrochloride (Camptosar®), BCG, live (Pacis®), leuprolide acetate implant (Viadur), bexarotene (Targretin®), exemestane (Aromasin®), topotecan hydrochloride (Hycamtin®), gemcitabine HCL(Gemzar®), daunorubicin hydrochloride (Daunorubicin HCL®), gemcitabine HCL (Gemzar®), toremifene citrate (Fareston), carboplatin (Paraplatin®), cisplatin (Platinol® and Platinol-AQ®) oxaliplatin and any other platinum-containing oncology drug, trastuzumab (Herceptin®), lapatinib (Tykerb®), gefitinb (Iressa®), cetuximab (Erbitux®), panitumumab (Vectibix®), temsirolimus (Torisel®), everolimus (Afinitor®), vandetanib (ZactimaTM), vemurafenib (ZelborafTM), crizotinib (Xalkori®), vorinostat(Zolinza®), bevacizumab (Avastin®), radiation therapy, hyperthermia, gene therapy and photodynamic therapy.

In the treatment of cancer, an appropriate dosage level of the therapeutic agent will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. The compounds may be administered on a regimen of 1 to 4 times per day, preferably once or twice per day. Administration by continuous infusion is also preferable.

Pharmaceutical preparation may be pre-packaged in ready-to-administer form, in amounts that correspond with a single dosage, appropriate for a single administration referred to as unit dosage form. Unit dosage forms can be enclosed in ampoules, disposable syringes or vials made of glass or plastic.

However, the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the patient undergoing therapy.

### EXAMPLES

Objective: Synthesize and optimize co-conjugated PEG:AS1411 gold nanoparticles (AuNPs) that exhibit GBM targeting abilities while allowing for the addition of a combinatorial treatment against glioblastoma cell types.

### Materials

HAuCl₄·3H₂O was purchased from Alfa Aesar (Tewksbury, Mass.). Citric acid, trisodium salt (Na₃C₆H₅O₇), sodium borohydride (NaBH₄), dithiothreitol (DTT) and anhydrous sodium bicarbonate (NaHCO₃) were purchased from Sigma Aldrich (St. Louis, Mo.). 10.0×phosphate buffered solution (pH 7.4). Nanopure ultrapure water (Barnstead, resistivity of 18.2 MΩ-cm) was used for preparing all aqueous solutions. Hydrochloric acid (HCl) and nitric acid (HNO₃) were analytical grades and purchased from VWR (Rednor, Pa.). Aqua regia solution (3 parts HCl and 1 part HNO₃), was used to clean all glassware for GNP synthesis. Thiol Polyethylene Glycol-4-alcohol (SH-PEG-OH; Molecular Weight 210.3 g/mol; 95% purity) was purchased from BroadPharm (San Diego, Calif.) and prepared via company specifications. Oligonucleotides having a regular DNA backbone (phosphodiester), a 5′-Thiol C₆ S-S modification (Thio-MC6-D), 5′-6T spacer (for AS1411 and CRO) and high-performance liquid chromatography purification were supplied by Integrated DNA Technologies. (Coralville, Iowa). The oligonucleotides sequences used were 5′-GGTGGTGGTGGTTGTGGTGGTGGTGG (AS1411; SEQ ID NO:10) and 5′-CCTCCTCCTCCTTCTCCTCCTCCTCC (CRO; SEQ ID NO:17), each modified with 6 thymine bases at the 5′ end and 3 thymine bases at the 3′ end. Fluorophore-modified oligonucleotides (Cy5-AS1411 and Cy5-CRO) are structurally similar oligonucleotides with a 3′ modified fluorescent Cyanine-5 (Cy5) were obtained from Integrated DNA Technologies. Illustra NAP-25 DNA size exclusion chromatography gravity columns were acquired from GE Healthcare Life Sciences (Pittsburgh, Pa.). Amicon Ultra 15.0 ml centrifugal filters with Ultracel-30 (30000 MWCO) were purchased from Merck Millipore (Billerica, Mass.). UV absorption spectra of nanoparticle formulations and oligos were measured with a UV Visible Spectrometer (Varian Cary 50 BIO UV, Agilent Technologies, Santa Clara, Calif.). Dynamic light scattering and zeta potential measurements were acquired on nanoparticle formulations using a NanoBrook Zeta PALS Zeta Potential Analyzer (Brookhaven Instruments, Holtsville, N.Y.). U87MG glioblastoma cancer cells were purchased from ATCC (Manassas, Va.). Dulbecco's Modified Eagle Medium, Heat Inactivated Fetal Bovine Serum, 10×Trypsin, Penicillin/Streptomycin were purchased from Thermo Fisher Scientific (Waltham, Mass.). GBM cells were subcultured in T25 or T75 sterile culture plates from Corning Incorporated (Tewksbury, Mass.). U87MGs were cultured in sterile culture plates from VWR (Radnor, Pa.) for Brightfield microscopy and metabolic studies or in 10 mm glass bottom, poly-d-lysine coated Matek dishes purchased from MaTek Life Sciences (Ashland, Mass.) for confocal studies. Metabolic activity on nanoparticle or control treated U87MGs was measured using spectrophotometry measurement of (2,3-Bis-(Methoxy-4-Nitro-5-Sulfophenyl-2H-Tetrazolium-5-Carboxanilide) (XTT) acquired from Biotum (Fremont, Calif.).

### Co-Conjugated AS1411 and PEG GNP Synthesis

4 nanometer (nm) citrate-capped GNPs were sterilely synthesized using previously reported protocols. [35,36] Thiol-modified AS1411 (SH-AS1411) contains disulfide linkages upon purchasing for storage and stability that were cleaved prior to conjugation via boiling the desired mixture of SH-AS1411, 1.0 M DTT, 0.25 M phosphate buffer (PB), and nanopure ultrapure water for 1 hour followed by cooling for another hour. SH-AS1411 was isolated via size exclusion chromatography using Illustra NAP-25 DNA gravity columns and 0.1M PB as the eluent. SH-PEG alcohol was diluted in DMSO according to manufacture specifications. Cleaved and purified SH-AS1411 and SH-PEG alcohol were then conjugated to GNPs in different ratios (see next paragraph) generating a two-component AS1411/PEG coating. Stepwise addition of 10×phosphate buffered saline (PBS) over a 96-hour period up to a concentration of 1×followed by maximum sonication in a water bath for 10 minutes. Centrifugation at 13,500 g for 20 minutes followed by a triplicate 1×-PBS washing and re-centrifugation after each wash removed any non-conjugated components.

Multiple PEG/AS1411 co-conjugated GNP formulations were synthesized that differed by their loading ratios of PEG and AS1411. Maximum possible loading of AS1411 onto GNPs was held constant at 12 times (expressed as 12×) the concentration of gold present within the colloidal solution, as measured by ultraviolet visible (UV-VIS) spectrometry. This 12×loading was divided up into multiple ratios of PEG:AS1411 and resulted in the following experimental conditions preserved throughout this paper: no PEG loading with maximum AS1411 loading (0:1 or 12×), maximum PEG loading with no AS1411 loading (1:0 or 0×), and three different loading ratios of AS1411:PEG (1:3 or 3×, 1:1 or 6×, and 3:1 or 9×). For formulations below the maximum of 12×AS1411, PEG was introduced to fulfill the 12×maximum loading requirements. Co-conjugated GNPs bearing SH-PEG and a CRO sequence as well as fluorescent co-conjugated and control GNPs (SH-AS1411-Cy5 or SH—CRO-Cy5, respectively, Exc./Em. 650/670) were synthesized similarly as stated above, once an optimal loading ratio was determined.

### Nanoparticle Characterization

Particle size (measured in nanometers), zeta potential (measured in millivolts), and polydispersity indices (unitless) were measured for all nanoparticle types from NanoBrook Zeta PALS Zeta Potential Analyzer (Brookhaven Instruments, Holtsville, N.Y.) running ZetaPALS software. Absorption spectra reported from Varian Cary 50 BIO UV (Agilent Technologies, Santa Clara, Calif.) spectrometer verified conjugations. Oligo loading was evaluated for each GNP formulation via a 72-hour cleavage of oligos from GNPs by treatment in a mixture of 1×PBS with 1.0 M DTT followed by UV-VIS measurement of cleaved and isolated oligos (via Illustra NAP-25 DNA gravity columns with 0.1 M phosphate buffer as the eluent). Moles of oligo were calculated from UV measurements using Beer's Law and compared to moles of gold present in colloidal solution to determine an average AS1411 or CRO per GNP.

### Cell Culture

U87MGs were cultured in Dulbecco's Modified Eagle's Medium (DMEM) with 10% heat inactivated fetal bovine serum and 1% penicillin/streptomyosin. All subcultures were passaged using 0.25% Trypsin and seeded at a minimum density of 5000 cells/cm². U87MGs were seeded at densities representing 1000 cells/well onto sterile clear polystyrene 96 well plates or onto 10 mm Matek glass bottom dishes. Cells seeded onto glass bottom dishes were allowed to adhere for 1 hour prior to culturing. U87MGs seeded onto plates or dishes were cultured for 2 days prior to GNP treatments to allow for cells to acclimate to culture conditions.

### Cytotoxicity and Specificity Studies

Cell and control wells in 96 well plates were incubated with nanoparticle formulations combined with DMEM ranging from 0-5000 micromolar (μM) AS1411 for 72 hours with no media changes. Control cells were treated with 10 μM AS1411, bare GNPs representing the highest gold concentration, or no treatment. XTT absorbance data was obtained from a Molecular Devices SpectraMax M2 Spectrometer running SoftMaxPro 7.0 software and was baseline corrected for GNP interference with the cell plates. Statistically significant cytotoxicity of GNP formulations on U87MGs was determined via two-way ANOVA analyses. Specificity of AS1411 was verified by comparing cytotoxicity of optimally determined co-conjugated GNPs bearing AS1411 to that of those bearing CRO. IC₅₀ (or the concentration at which effectively inhibits 50% survival) analyses using GraphPad Prism version 7.0.0 for Windows, GraphPad Software, (San Diego, Calif.; www.graphpad.com) determined effectiveness of optimal co-conjugated GNPs.

### Microscopy Studies

10× brightfield images were acquired in 96 well plates using a Nikon TE200 Epiflorescent microscope (Melville, N.Y.) with a Coolsnap HQ CCD camera (Roper, Duluth, Ga.) enabled with NIS Elements software on U87MGs treated with GNP formulations and controls 72 hours prior to XTT treatments. Cellular uptake and cytotoxicity of co-conjugated Cy5-AS1411 or Cy5-CRO GNPs was verified using a Nikon A1R Spectral Confocal microscope fitted with live cell chambers. U87MGs seeded onto Matek dishes were treated at previously determined IC₅₀ reported from cytotoxicity studies and imaged under 20× magnification for 72 hours at a rate of 1 image per 30 minutes. Images were acquired from 5 separate xy-positions for each condition. Using Nikon Elements software, images were then converted to .avi videos and observed for distribution of particles. Representative photos were chosen for 0, 24, 48, and 72 hours for all conditions.

### Statistical Analysis

All data were collected and processed in Microsoft Excel. Appropriate statistical tests were completed using Graphpad Prism using a significance level of α=0.05. Data is presented as mean values+/−standard deviation. Statistical tests reported are two-way ANOVAs with Bonferroni post-hoc tests. Sample sizes are described where needed.

### Results

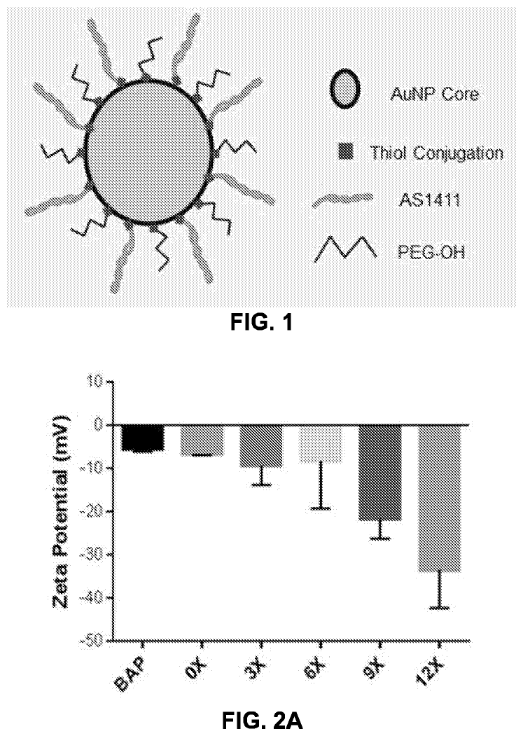

To determine the optimal configuration of PEG-AS1411-GNPs for GBM applications, human GBM-representing cell lines (U87MGs) were exposed to GNPs with differing loading ratios of PEG and AS1411 components. Multiple PEG/AS1411 co-conjugated GNP formulations were synthesized that differed by their loading ratios of PEG and AS1411. PEG is an anti-fouling molecule, thus protecting particles from protein aggregation, and is used as a surface modifier to enable further modification of GNPs with additional anti-GBM therapies. Maximum possible loading of AS1411 and PEG onto GNPs was held constant at 12 times (expressed as 12×) the concentration of gold present within colloidal solution, as measured by UV-VIS spectrometry. This 12×loading was divided up into multiple ratios of PEG:AS1411 and resulted in the following experimental conditions preserved throughout the results: no PEG loading with maximum AS1411 loading (0:1 or 12×), maximum PEG loading with no AS1411 loading (1:0 or 0×), and three different loading ratios of AS1411:PEG (1:3 or 3×, 1:1 or 6×, and 3:1 or 9×). For formulations below the maximum of 12×AS1411, PEG was introduced to fulfill the maximum loading requirements. A schematic of the GNPs (FIG. 1) shows the proposed topographic features. Cytotoxicity and brightfield imaging were then completed and taken in conjunction with particle synthesis characteristics to determine the best particle design for GBM applications. To verify retention of AS1411's specificity on optimized particles, cytotoxicity studies were completed with optimal GNP formulations bearing either AS1411 or CRO, a control oligonucleotide sequence. Live cell imaging studies were then completed verifying the overall uptake and distribution of optimally designed PEG-AS1411-GNPs.

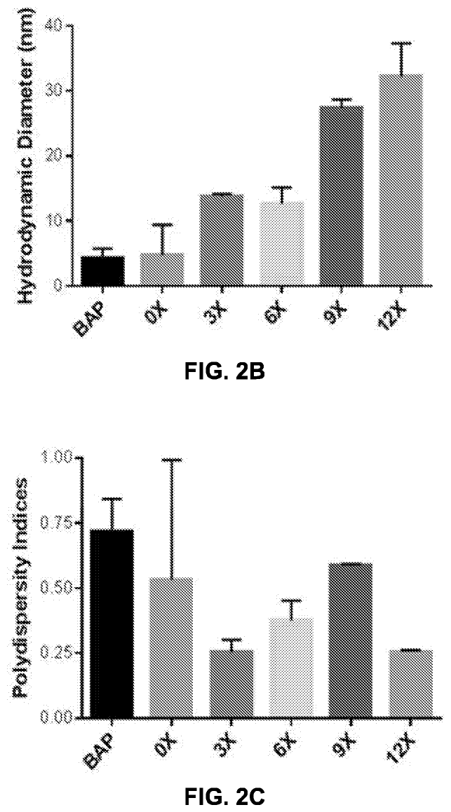

Zeta Potential (mV) and hydrodynamic size (nm) measurements (mV) are shown in FIG. 2A and FIG. 2B, respectively. Magnitudes larger than 10 indicate more stable syntheses. Data shows minimal size variation.

Polydispersity indices is shown in FIG. 2C. Polydispersity is unitless, and measured from 0 to 1. AS1411 per AuNP is shown in FIG. 2D.

DTT causes separation of conjugated AS1411. UV-VIS spectral analysis of before and after separation are shown in FIG. 2E and FIG. 2F, respectively. UV-VIS spectra show gold's surface plasmon resonance (˜520 nm) and DNA (˜260 nm) verified loading.

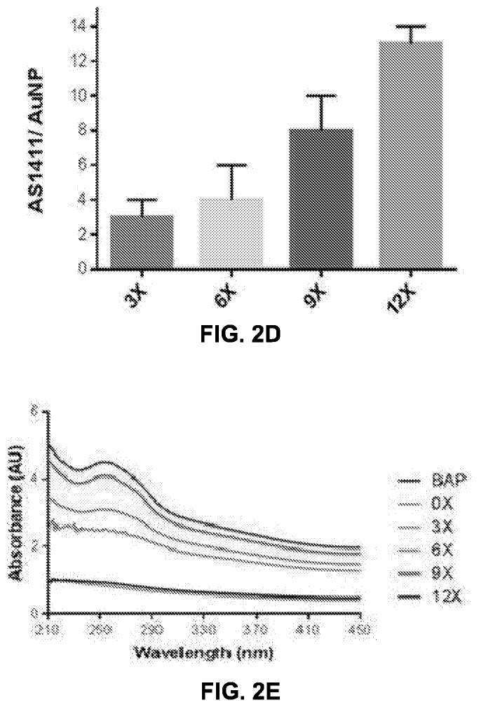

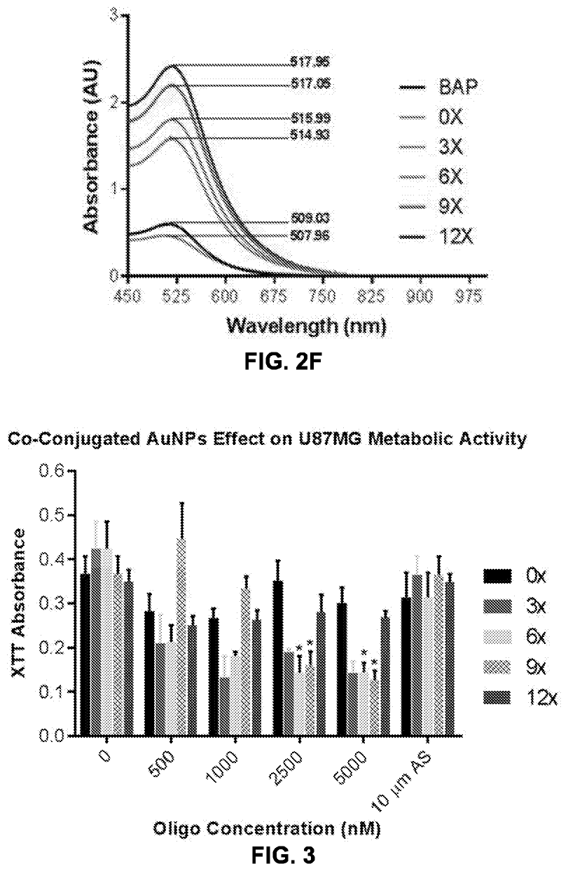

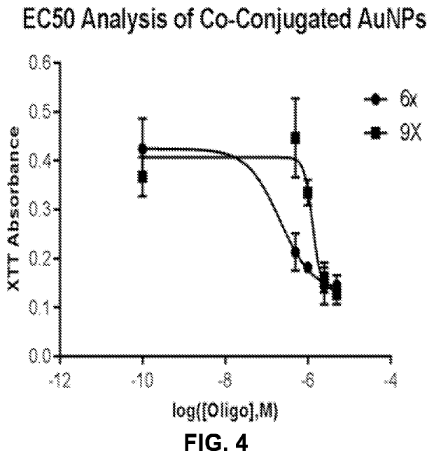

Co-conjugated (AS1411 and PEG) AuNPs effect on U87MG cells metabolic activity is shown in FIG. 3. EC50 analysis of co-conjugated AuNPs on the same cells is shown in FIG. 4. 10× brightfield images of particle treated U87MG cells is shown in FIG. 5. The lower the XTT absorbance, the lower the metabolic activity and the greater the cell killing effect.

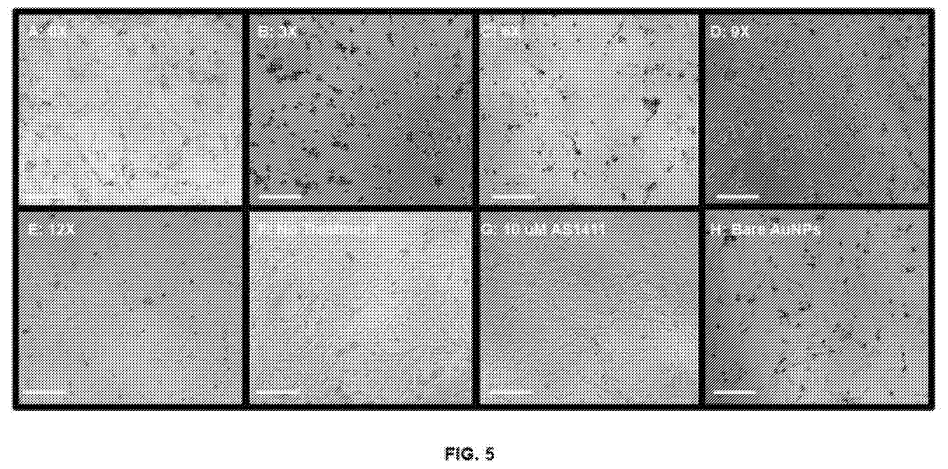

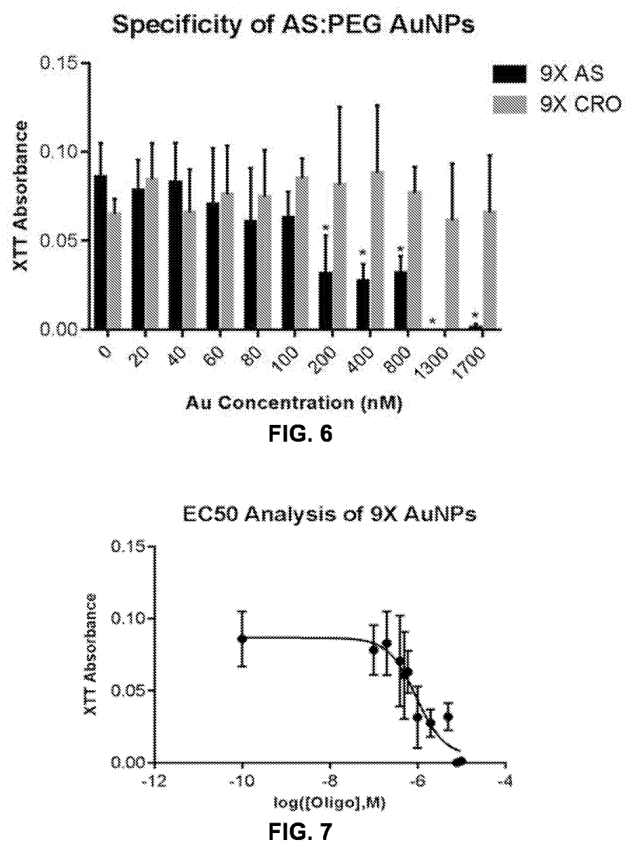

In-vitro metabolic activity of U87MGs after treatment with 9×AS/CRO AuNPs is shown in FIG. 6. FIG. 7 is a graph showing EC50 analysis of 9×co-conjugated AuNP particles.

The results demonstrate a surprising and unexpected improvement of anti-nucleolin agent-PEG-conjugated nanoparticles as compared to anti-nucleolin agent-conjugated nanoparticles. Particularly at 2500 nm and 5000 nm concentrations of AS1411, all forms of anti-nucleolin agent-PEG-conjugated nanoparticles were nearly twice as effective at cell killing as compared to anti-nucleolin agent-conjugated nanoparticles, as shown in FIG. 3. This is surprising and unexpected, because the benefits of PEGylation would only have been expected in vivo, not in vitro as shown here. Furthermore, live cell confocal studies have verified the selectivity of anti-nucleolin agent-PEG-conjugated nanoparticles. Similar results would be expected in other types of cancer.

### REFERENCES

1. Hendrik Heinz, Nanoparticle decoration with surfactants: Molecular interactions, assembly, and applications; Surface Science Reports, Volume 72, Issue 1, 2017, Pages 1-5
2. Singh, Priyanka et al, Biological Synthesis of Nanoparticles from Plants and Microorganisms; Trends in Biotechnology, Volume 34, Issue 7, 588-599
3. Ding, Y., et al., Gold Nanoparticles for Nucleic Acid Delivery. Molecular Therapy, 2014. 22(6): p. 1075-1083.
4. Roca, M. and A. J. Haes, Probing cells with noble metal nanoparticle aggregates. Nanomedicine, 2008. 3(4): p. 555-565.
5. DeLong, R. K., et al., Functionalized gold nanoparticles for the binding, stabilization, and delivery of therapeutic DNA, RNA, and other biological macromolecules. Nanotechnol Sci Appl, 2010. 3: p. 53-63.
6. Sperling, R. A., et al., Electrophoretic Separation of Nanoparticles with a Discrete Number of Functional Groups. Advanced Functional Materials, 2006. 16(7): p. 943-948.
7. Asian, K., C. C. Luhrs, and V. H. Perez-Luna, Controlled and Reversible Aggregation of Biotinylated Gold Nanoparticles with Streptavidin. The Journal of Physical Chemistry B, 2004. 108(40): p. 15631-15639.
8. Gole, A. and C. J. Murphy, Azide-derivatized gold nanorods: functional materials for “click” chemistry. Langmuir, 2008. 24(1): p. 266-72.
9. Brennan, J. L., et al., Bionanoconjugation via click chemistry: The creation of functional hybrids of lipases and gold nanoparticles. Bioconjug Chem, 2006. 17(6): p. 1373-5
10. Goldshmit, Y., et al., Interfering with the interaction between ErbB1, nucleolin and Ras as a potential treatment for glioblastoma. Oncotarget, 2014. 5(18): p. 8602-13.
11. Benedetti, E., et al., Nucleolin antagonist triggers autophagic cell death in human glioblastoma primary cells and decreased in vivo tumor growth in orthotopic brain tumor model. Oncotarget, 2015. 6(39): p. 42091-104.
12. Abdelmohsen, K. and M. Gorospe, RNA-binding protein nucleolin in disease. RNA Biology, 2012. 9(6): p. 799-808.
13. Bates, P. J., et al., Discovery and development of the G-rich oligonucleotide AS1411 as a novel treatment for cancer. Exp Mol Pathol, 2009. 86(3): p. 151-64.
14. Malik, M. T., et al., AS1411-conjugated gold nanospheres and their potential for breast cancer therapy. Oncotarget, 2015. 6(26): p. 22270-81.
15. Laber D, T. B., Kloecker G, Bates P, Trent J, Miller D. Extended phase I study of AS1411 in renal and non-small cell lung cancers. in ASCO Annual Meeting Proceedings. J Clin Oncol.
16. Stuart R, S.-G. K., Cooper M, Devetten M, Herzig R, Medeiros B, Schiller G, Wei A, Acton G, Rizzieri D. Randomized phase II trial of the nucleolin targeting aptamer AS1411 combined with high-dose cytarabine in relapsed/refractory acute myeloid leukemia (AML). in ASCO Annual Meeting Proceedings (Post-Meeting Edition). Journal of Clinical Oncology.
17. Kwak, H. J., et al., Downregulation of Spry2 by miR-21 triggers malignancy in human gliomas. Oncogene, 2011. 30(21): p. 2433-42.
18. Kyriazi, M.-E., et al., Multiplexed mRNA Sensing and Combinatorial-Targeted Drug Delivery Using DNA-Gold Nanoparticle Dimers. ACS Nano, 2018. 12(4): p. 3333-3340.
19. Deng, R., et al., Targeting epigenetic pathway with gold nanoparticles for acute myeloid leukemia therapy. Biomaterials, 2018. 167: p. 80-90.
20. Deorah, S., et al., Trends in brain cancer incidence and survival in the United States: Surveillance, Epidemiology, and End Results Program, 1973 to 2001. Neurosurg Focus, 2006. 20(4): p. E1.
21. Mujokoro., B., Nano-structures mediated co-delivery of therapeutic agents for glioblastoma treatment: A review. In Materials Science and Engineering 2016.
22. QT., O., CBTRUS Statistical Report: Primary Brain and Central Nervous System Tumors Diagnosed in the United States in 2008-2012. Neuro. Oncol., 2015.
23. Reardon, D. A., et al., Bevacizumab continuation beyond initial bevacizumab progression among recurrent glioblastoma patients. British Journal of Cancer, 2012. 107(9): p. 1481-1487.
24. Zhang Y, C. N., Pahuski M, et al., Noncoding RNAs in Glioblastoma. In: De Vleeschouwer S, editor. 2017.
25. Tamimi A F, J. M., Epidemiology and Outcome of Glioblastoma. In: De Vleeschouwer S, editor. Glioblastoma [Internet]. 2017 Sep. 27.
26. Stupp, R., et al., Radiotherapy plus concomitant and adjuvant temozolomide for glioblastoma. N Engl J Med, 2005. 352(10): p. 987-96.
27. Ahmed, R., et al., Malignant gliomas: current perspectives in diagnosis, treatment, and early response assessment using advanced quantitative imaging methods. Cancer Manag Res, 2014. 6: p. 149-70.
28. Wadajkar, A. S., et al., Tumor-targeted nanotherapeutics: overcoming treatment barriers for glioblastoma. Wiley Interdiscip Rev Nanomed Nanobiotechnol,
29. Fernandes, C., et al., Current Standards of Care in Glioblastoma Therapy, in Glioblastoma, S. De Vleeschouwer, Editor. 2017, Codon Publications
30. Hart, M. G., et al., Temozolomide for high grade glioma. Cochrane Database of Systematic Reviews, 2013(4).
31. Lee, Y. S. and A. Dutta, MicroRNAs in cancer. Annu Rev Pathol, 2009. 4: p. 199-227.
32. Moller, H. G., et al., A systematic review of microRNA in glioblastoma multiforme: micro-modulators in the mesenchymal mode of migration and invasion. Mol Neurobiol, 2013. 47(1): p. 131-44.
33. Shea, A., et al., MicroRNAs in glioblastoma multiforme pathogenesis and therapeutics. Cancer Med, 2016. 5(8): p. 1917-46.
34. Comprehensive genomic characterization defines human glioblastoma genes and core pathways. Nature, 2008. 455(7216): p. 1061-8.
35. Kim, H., et al., Integrative genome analysis reveals an oncomir/oncogene cluster regulating glioblastoma survivorship. Proc Natl Acad Sci USA, 2010. 107(5): p. 2183-8.
36. Li, Y., et al., MicroRNA-34a inhibits glioblastoma growth by targeting multiple oncogenes. Cancer Res, 2009. 69(19): p. 7569-76.
37. Bandman O, Yue H, Corley N C, Shah P, “Human Nucleolin-like Protein” U.S. Pat. No. 5,932,475 (3 Aug. 1999).
38. Bates P J, Miller D M, Trent J O, Xu X, “A New Method for the Diagnosis and Prognosis of Malignant Diseases” International Application, Int'l Pub. No. WO 03/086174 A2 (23 Oct. 2003).
39. Bates P J, Miller D M, Trent J O, Xu X, “Method for the Diagnosis and Prognosis of Malignant Diseases” U.S. Patent App. Pub., Pub. No. US 2005/0053607 A1 (10 Mar. 2005).
40. Bates P J, Kahlon J B, Thomas S D, Trent J O , Miller D M, “Antiproliferative Activity of G-rich Oligonucleotides Correlates with Protein Binding” J. Biol. Chem. 274:26369-77 (1999).
41. Reyes-Reyes E M, Teng Y, Bates P J, “A New Paradigm for Aptamer Therapeutic AS1411 Action: Uptake by Macropinocytosis and Its Stimulation by a Nucleolin-Dependent Mechanism” Cancer Res 70(21): 8617-29 (2010).
42. Stedman, T. L. 2000. Stedman's medical dictionary. Lippincott Williams & Wilkins, Philadelphia. xxxvi, [127], 2098.
43. Miller D M, Bates P J, Trent J O , Xu X, “Method for the Diagnosis and Prognosis of Malignant Diseases” U.S. Patent App. Pub., Pub. No. US 2003/0194754 A1 (16 Oct. 2003).
44. Cooper et al. [2014], Front. Chem. 14, 2, 86.
45. Alkilany, A. M. and C. J. Murphy, Toxicity and cellular uptake of gold nanoparticles: what we have learned so far? Journal of Nanoparticle Research, 2010. 12(7): p. 2313-2333.
46. Guo, J. et al., Aptamer-functionalized PEG-PLGA nanoparticles for enhanced anti-glioma drug delivery. Biomaterials 32 (2011) 8010-8020.
47. Takafuji, Y., Jo, J-I and Tabata, Y. Simple PEG Modification of DNA Aptamer Based on Copper Ion Coordination for Tumor Targeting. J. Biomaterials Science 22 (2011) 1179-1195.
48. Aravind, A. et al., AS1411 Aptamer Tagged PLGA-Lecithin-PEG Nanoparticles for Tumor Cell Targeting and Drug Delivery. Biotechnology and Bioengineering 109(11) pp. 2920-2931 (November 2012).
49. Malik, M. T., et al. AS1411-conjugated gold nanospheres and their potential for breast cancer therapy. Oncotarget 6(26) pp. 22270-22281 (2015).

1. A composition, comprising an anti-nucleolin agent and PEG conjugated to nanoparticles, wherein the nanoparticles have an average diameter of 1 to 50 nm.

2. A composition, comprising an anti-nucleolin agent and PEG conjugated to nanoparticles, wherein the nanoparticles comprise at least one inorganic material selected from the group consisting of metals, elements and oxides.

3. The composition of claim 1, wherein the nanoparticles comprise gold.

4. The composition of claim 1, wherein the anti-nucleolin agent comprises an anti-nucleolin oligonucleotide.

5. The composition of claim 1, wherein the anti-nucleolin agent comprises an antibody.

6. The composition of claim 1, wherein the anti-nucleolin agent comprises a nucleolin targeting protein.

7. The composition of claim 1, wherein the anti-nucleolin agent comprises a GRO.

8. (canceled)

9. The composition of claim 1, wherein the anti-nucleolin agent comprises AS1411.

10. The composition of claim 1, further comprising a cyanine dye.

11. (canceled)

12. The composition of claim 1, wherein the nanoparticles have an average diameter of 1 to 20 nm.

13. The composition of claim 1, wherein the nanoparticles comprise at least one metal selected from the group consisting of gold, platinum, iridium and palladium.

14. The composition of claim 1, further comprising gadolinium.

15. (canceled)

16. The composition of claim 14, wherein the gadolinium is present as a complex of gadolinium (Ill).

17. (canceled)

18. A method of treating cancer, comprising administering an effective amount of the composition of claim 1, to a patient in need thereof.

19. A method of treating cancer, comprising administering an effective amount of the composition of claim 1, to a patient in need thereof, followed by radiation therapy.

20. The method of treating cancer of claim 18, wherein the cancer is selected from the group consisting of melanoma, lymphoma, plasmocytoma, sarcoma, glioma, glioblastoma, thymoma, leukemia, breast cancer, prostate cancer, colon cancer, liver cancer, esophageal cancer, brain cancer, lung cancer, ovary cancer, endometrial cancer, bladder cancer, kidney cancer, cervical cancer and hepatoma.

21. The method of treating cancer of claim 18, wherein the cancer is selected from the group consisting of breast cancer, prostate cancer, colon cancer, glioblastoma and lung cancer.

22. The method of treating cancer of claim 18, further comprising administering a second cancer treatment selected from the group consisting of vinorelbine, mytomycin, camptothecin, cyclyphosphamide, methotrexate, tamoxifen citrate, 5-fluorouracil, irinotecan, doxorubicin, flutamide, paclitaxel, docetaxel, vinblastine, imatinib mesylate, anthracycline, letrozole, arsenic trioxide, anastrozole, triptorelin pamoate, ozogamicin, irinotecan hydrochloride, BCG live, leuprolide acetate implant (Viadur), bexarotene, exemestane, topotecan hydrochloride, gemcitabine HCL, daunorubicin hydrochloride, gemcitabine HCL, toremifene citrate (Fareston), carboplatin, cisplatin, oxaliplatin and any other platinum-containing oncology drug, trastuzumab, lapatinib, gefitinb, cetuximab, panitumumab, temsirolimus, everolimus, vandetanib, vemurafenib, crizotinib, vorinostat, bevacizumab, radiation therapy, hyperthermia, gene therapy and photodynamic therapy.

23. A method of imaging cancer by MRI and/or X-ray imaging techniques in vivo, comprising:
administering the composition of claim 1, to a subject and
forming an image of the composition present in the subject by MRI and/or an X-ray imaging technique.

24. The method of imaging cancer of claim 24, wherein the administering occurs more than 24 hours before the forming.
